SEQUENCE LISTING (14 sequences)

SEQ ID NO: 1 - protein, 15 aa, Artificial Sequence
  Description of Artificial Sequence Synthetic peptide
  Biotinylation tag
GLNDIFEAQKIEWHE

SEQ ID NO: 2 - protein, 296 aa, Artificial Sequence
  Description of Artificial Sequence Synthetic polypeptide
  Biotinylation-tagged HLA-E
MGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPGSGGGLNDIFEAQKIEWHE

SEQ ID NO: 3 - protein, 100 aa, Homo sapiens [sequence omitted]

SEQ ID NO: 4 - protein, 9 aa, Mycobacterium tuberculosis
RLPAKAPLL

SEQ ID NO: 5 - protein, 9 aa, Mycobacterium tuberculosis
ILPSDAPVL

SEQ ID NO: 6 - protein, 9 aa, Mycobacterium tuberculosis
VMATRRNVL

SEQ ID NO: 7 - protein, 9 aa, Human immunodeficiency virus
RMYSPTSIL

SEQ ID NO: 8 - protein, 9 aa, Human immunodeficiency virus
RMYSPVSIL

SEQ ID NO: 9 - protein, 9 aa, Homo sapiens
VMAPRTLIL

SEQ ID NO: 10 - protein, 9 aa, Homo sapiens
VMAPRTLVL

SEQ ID NO: 11 - protein, 9 aa, Artificial Sequence
  Description of Artificial Sequence Synthetic peptide
  Modified Mycobacterium tuberculosis
  MOD_RES (9)..(9): L-3-C homocysteine
RLPAKAPLC

SEQ ID NO: 12 - protein, 9 aa, Artificial Sequence
  Description of Artificial Sequence Synthetic peptide
  Modified Mycobacterium tuberculosis
  MOD_RES (9)..(9): L-4-C homocysteine
RLPAKAPLC

SEQ ID NO: 13 - protein, 4 aa, Artificial Sequence
  Description of Artificial Sequence Synthetic peptide
GSGG

SEQ ID NO: 14 - protein, 12 aa, Artificial Sequence
  Description of Artificial Sequence Synthetic peptide
RLPAKAPLLGCG

(19) United States
(12) Patent Application Publication
(10) Pub. No.: US 2023/0054274 A1
(43) Pub. Date: Feb. 23, 2023

(54) PEPTIDE-MHC COMPLEXES

(71) Applicant: IMMUNOCORE LIMITED, Abingdon, Oxfordshire (GB)

(72) Inventors: Thomas Holberg BLICHER, Abingdon, Oxfordshire (GB); Victoria Arena DE SOUZA, Abingdon, Oxfordshire (GB)

(21) Appl. No.: 17/624,222

(22) PCT Filed: Jul. 1, 2020
(86) PCT No.: PCT/EP2020/068491

§ 371 Date: Dec. 30, 2021

(30) Foreign Application Priority Data
Jul. 2, 2019 (GB) 1909509.0

(51) Int. Cl. C07K 14/74; G01N 33/50; G01N 33/68

(52) U.S. Cl. CPC C07K 14/70539; G01N 33/505; G01N 33/68

(57) ABSTRACT

The present invention provides a stabilised peptide-MHC (pMHC) complex, such as a peptide-HLA-E complex. The complex has a non-native linkage, such as a disulphide bond, between the C terminal anchor residue of the peptide, and an amino acid residue in the F pocket of the MHC binding groove.

The present invention relates to peptide-MHC (pMHC) complexes. In particular, it relates to pMHC complexes that are stabilised and that retain native-like TCR recognition.

MHC molecules play a critical role in immune surveillance by presenting endogenous and exogenous antigenic peptides to T cells. MHC class I complexes are composed of two subunits, a heavy chain that consists of three extracellular domains (α1, α2 and α3), a transmembrane domain and a cytoplasmic tail, and a light chain termed β2 Microglobulin (β2M), which is required for the expression of all Class I molecules in vivo. The α1 and α2 domains of the heavy chain together are structurally arranged to form a platform of eight anti-parallel β stands flanked by two alpha helices, which forms the peptide binding groove. MHC class I molecules typically bind peptides of approximately 8 to 10 amino acids. MHC Class II complexes share a similar overall architecture to Class I complexes, but the peptide binding groove is formed from both subunits and is in a more open configuration to accommodate longer peptides, typically in the range of 11-30 amino acids. The binding groove of MHC complexes can be considered to be divided into six pockets or subsites, designated pockets A to F. The pockets at each end of the binding groove (A and F) are highly conserved and are responsible for binding the N and C terminal anchor residues of peptide through extensive hydrogen bonding networks. The other pockets are polymorphic and therefore play a role in determining the specificity of peptide interaction (Matsumura et al., Science. 1992 Aug. 14; 257(5072):927-34).

Isolated pMHC complexes are essential tools in immunology research and in the development of various therapeutic modalities; however, in some cases peptides bind weakly to MHC and thus rapidly dissociate. This can pose a challenge for methodologies that rely on stable complexes, such as enumerating T cell responses with MHC multimers. pMHC complex instability appears to be particularly problematic for peptides that bind to the non-classical human MHC class I molecule, HLA-E.

Unlike classical MHC molecules, HLA-E exists almost exclusively in just two allelic forms, E*01:01 and E*01:03, which differ by just one amino acid. For this reason, peptides presented by HLA-E make attractive targets for immunotherapy, as they circumvent the challenges inherent in targeting the highly polymorphic, classical MHC molecules. Under normal conditions, HLA-E binds and presents leader sequence peptides, cleaved from other HLA class I molecules, to NK cells as a method of immune surveillance. Defects in antigen processing machinery, caused by certain infectious agents or in tumour tissues, lead to targeted killing by NK cells, and are associated with an increase in the HLA-E peptide repertoire, potentially enabling T-cell immunosurveillance. There is increasing evidence that HLA-E can present a number of bacterial and viral peptides, such as from Mycobacterium tuberculosis or HIV, and that these peptide HLA-E complexes are capable of stimulating CD8⁺ T-cells (van Hall et al., Microbes Infect. 2010 November; 12(12-13):910-8; Joosten et al., PLoS Pathog. 2010 Feb. 26; 6(2):e1000782; Joosten et al., J Immunol Res. 2016; 2016:2695396; Hansen et al., Science. 2016 Feb. 12; 351(6274):714-20; Nattermann et al., Antivir Ther. 2005; 10(1):95-107; Nattermann et al., Am J Pathol. 2005 February; 166(2):443-53). The role of HLA-E appears to be conserved across a range of mammals, including primates (Wu et al., J Immunol. 2018 Jan. 1; 200(1):49-60) and mice (Oliveira et al., J Exp Med. 2010 Jan. 18; 207(1):207-21). Efforts to exploit immunotherapeutic approaches that target peptide HLA-E complexes have been hampered by the poor stability of isolated peptide HLA-E complexes compared to classical class I complexes such as HLA-A*02. For example, instability of the isolated peptide HLA-E complex can hamper the identification and subsequent development of T cell receptor (TCR) or antibody-based therapeutics that specifically recognise the complex.

Methods to stabilise isolated pMHC complexes are known in the art (for example see U.S. Pat. No. 8,992,937; WO2013030620; Truscott J Immunol. 2007 May 15; 178(10):6280-9; Mitaksov et al., Chem Biol. 2007 August; 14(8):909-22). However, the inventors have found that such approaches are not suitable for stabilising pMHC complexes, including those comprising HLA-E, in a way that retains native like TCR binding. The present invention aims to provide stabilised peptide-MHC complexes that demonstrate native-like TCR recognition.

In a first aspect, the present invention provides a stabilised peptide-MHC (pMHC) complex, comprising a non-native linkage between the C terminal anchor residue of the peptide, and an amino acid residue in the F pocket of the MHC binding groove.

The inventors have unexpectedly found that pMHC complexes can be stabilised by the introduction of a non-native linkage between the C terminal anchor amino acid residue of the peptide, and an amino acid residue in the F pocket of the MHC binding groove. The linkage is such that the pMHC complex retains the three-dimensional conformation of the native pMHC complex and can be recognised by a TCR that recognises the native complex.

pMHC complexes of the invention are stabilised in the sense that they have a superior stability relative to the native complex that does not have the non-native linkage between the C terminal anchor residue of the peptide, and an amino acid residue in the F pocket of the MHC binding groove. Stability may be assessed by Surface Plasmon Resonance (SPR) or Bio-layer interferometry (BLI) methods well known to a person skilled in the art, such as Biacore or Octet, respectively. pMHC complexes in accordance with the invention will have a binding half life that is longer than that of the native pMHC complex. The binding half life of the pMHC complexes of the present invention may be at least 2 times, at least 3 times, at least 4 times or at least 5 times greater than the binding half life of the native pMHC complex. In pMHC complexes of the present invention, the peptide may have a binding half life to MHC of at least 3 hours. Preferably, the binding half life is at least 4 hours, at least 5 hours, at least 10 hours, at least 15 hours, or at least 20 hours. Alternative approaches to determine complex stability include thermal denaturation.

pMHC complexes of the present invention retain the native three-dimensional conformation of the native pMHC complex. Accordingly, they may be recognised by a peptide MHC binding moiety, such as a TCR, or a TCR-mimic antibody, that recognises the native complex. Recognition may be determined by SPR. The affinity of the binding moiety for the pMHC complex of the present invention may be less than 3-fold different to the affinity of the binding moiety for the native complex, when measured under comparable conditions. Those skilled in the art will appreciate that certain native pMHC complexes are so unstable (e.g. have such a short binding half life) under standard conditions (such as room temperature) that it may be impossible to measure the affinity of the binding moiety for the native complex and thereby make a comparison to the affinity of the binding moiety for the complex of the present invention. In these circumstances, it may be possible to measure—and compare—affinity by changing the conditions, such as lowering the temperature. The binding moiety may have a K_{D} for the complex of at least 100 μM, at least 50 μM, at least 10 μM, at least 1 μM or stronger, under standard conditions, such as those set out in Example 2B.

The pMHC complexes of the present invention comprise a non-native linkage between the C terminal anchor residue of the MHC binding peptide and an amino acid residue in the F pocket of the peptide binding groove. This linkage is such that the peptide is stabilised in the binding groove. The non-native linkage does not perturb the conformation of the peptide in the binding groove, meaning that it should be recognised in a native-like manner by peptide MHC binding moieties, such as TCRs. Peptide conformation may be determined by x-ray crystallography. The linkage may be a covalent bond. The covalent bond may be formed between amino acids substituted for amino acid residues in the F pocket of the pMHC binding groove and/or the C terminal anchor residue of the peptide, preferably in both the F pocket of the MHC binding groove and the C terminal anchor residue of the peptide. At least one of the amino acids substituted for amino acid residues in the F pocket of the MHC binding groove and/or the C terminal anchor residue may be a non-natural amino acid. It is preferred if the C terminal anchor residue is a non-natural amino acid. The skilled person is aware of which amino acid positions of an MHC molecule are located in the F pocket (see for example table I in Matsumura et al., Science. 1992 Aug. 14; 257(5072):927-34). Preferably, the linkage is between the C terminal anchor residue of the MHC binding peptide and an amino acid residue at position 116 of the MHC heavy chain. Alternatively, also preferred is a linkage between the C terminal anchor residue of the MHC binding peptide and an amino acid residue located at position 147 of the MHC heavy chain. Other suitable locations on the MHC heavy chain for the linkage include positions 81, 124 and 143.

Preferably, the non-native linkage is a disulphide bond. This bond may be formed between amino acids that are able to form a disulphide bond (such as cysteine or a derivative thereof) substituted for amino acid residues in the F pocket of the MHC binding groove and/or the C terminal anchor residue of the peptide, preferably in both the F pocket of the MHC binding groove and the C terminal anchor residue of the peptide.

The table below indicates the identity and location of preferred cysteine or a derivative thereof mutations in various MHC Class I molecules. The numbering refers to the position on the MHC heavy chain.

| | | | HLA-A11 | HLA-A24 | | |
|---|---|---|---|---|---|---|
| | HLA-A2 | HLA-A3 | (A*1101/ | (A*240/2 | | |
| HLA-E | (A2*0201) | (A*0301) | A*1102) | A*2407) | HLA-F | HLA-G |
| L81 | L81 | L81 | L81 | A81 | L81 | L81 |
| F116 | Y116 | D116 | D116 | Y116 | H116 | Y116 |
| S143 | T143 | T143 | T143 | T143 | T143 | S143C |
| L124 | I124 | I124 | I124 | I124 | I124 | L124 |
| S147 | W147 | W147 | W147 | W147 | Y147 | C147 |

Most preferred mutations are cysteine or a derivative thereof substitutions at position 116 or position 147. Particularly preferred mutations include cysteine or a derivative thereof substitutions at position F116 or position S147 in HLA-E. Alternative positions for cysteine or a derivative thereof substitutions in HLA-E are L81, S143 or L124.

The pMHC complex may comprise two MHC subunits, a heavy chain and a light chain. The MHC subunits associate with a peptide ligand, which binds to the binding groove formed by one, or both, subunits. Preferably, the MHC complex is a Class I MHC complex. Alternatively, the MHC complex is be a Class II MHC complex. The MHC complex may be soluble. Methods for producing soluble complexes are known in the art; for example, the heavy chain of a Class I complex may be truncated to remove the transmembrane and cytoplasmic regions. Preferably, the MHC complex is from human and may be termed Human Leukocyte Antigen (HLA) complex instead of MHC. Alternatively, the MHC complex may be from other species, such as mouse or non-human primates. The MHC complex may be classical or non-classical. Classical MHC complexes from human include the polymeric HLA-A, HLA-B, and HLA-C. Non-classical MHC complexes that present peptide ligands include HLA-E, HLA-F and HLA-G. Preferably, the MHC complex is the non-classical HLA-E.

The pMHC complex may contain one or more mutations within in the MHC subunits relative to a natural MHC complex. Mutations include substitutions, insertions and deletions. Preferably, mutations are made at one or more positions within the F pocket of the peptide binding groove. Alternatively or additionally, mutations, including insertions, substitutions or deletions may be made at other locations within the MHC, provided they do not interfere with the stability of the isolated complex or recognition by binding moieties. Mutations in the F pocket of the binding groove include substitution of one or more amino acids to a cysteine.

The pMHC complex comprises a peptide ligand, which may be termed an MHC binding peptide. The MHC binding peptide may be 8 to 30 amino acids in length. The peptide may be 8, 9, 10, 11, 12, 13 or 14 amino acids, or longer, in length. Preferably the MHC binding peptide is 9 amino acids in length. The MHC binding peptide may have an amino acid sequence that corresponds to that of a natural MHC binding peptide sequence. Alternatively, the MHC binding peptide may contain one or more mutations relative to the amino acid sequence of a natural MHC binding peptide. Mutations may include substitutions, insertions and deletions. Preferably, the MHC binding peptide is a HLA-E binding peptide. Alternatively, or additionally, the MHC binding peptide may bind to other MHC complexes. There are many examples of known MHC binding peptides in the art. MHC binding peptides may be derived from exogenous proteins, including viral or bacterial proteins, or may be derived from endogenous self proteins. Methods to identify MHC binding peptides are known in the art, for example using in silico predictions (such as SYFPEITHI, (Rammensee et al., Immunogenetics (1999) 50: 213-219) and NetMHCpan (Jurtz et al., J Immunol. 2017 Nov. 1; 199(9):3360-3368)) and/or using mass spectrometry to identify peptide MHC complexes eluted from the cell surface.

The MHC binding peptide may contain a mutation at one or more of the positions involved in binding to MHC. As is known to those skilled in the art, MHC binding peptides contain anchor residues, which are involved in stabilising the interaction between the peptide and the MHC binding groove. The binding groove of MHC Class I is closed at both ends by conserved tyrosine residues leading to a size restriction of the bound peptides to usually 8-10 residues, with the C-terminal end of the peptide docking into the F-pocket of the MHC. The position and identity of anchor residues are known (Falk et al., Nature. 1991 May 23; 351(6324):290-6). For example, the anchor residues of peptides that bind to HLA-A2 are located at positions 2 and 9. A similar position for anchor residues has been found for HLA-E binding peptides (Lampen et al., Mol Immunol. 2013 January; 53(1-2):126-31). Typically the identity of the amino acids at the anchor positions is fixed, or shows limited variation. For all MHC binding peptides, the C terminal anchor residue is hydrophobic and its side chain is accommodated within the deep hydrophobic F pocket of the MHC binding groove. Preferably, in the present invention, the MHC binding peptide is mutated at the C terminal anchor residue (denoted P9 or PΩ). Where the MHC binding peptide binds to HLA-E, the C terminal anchor residue may be at position 9 of the peptide. Peptides that bind to HLA-E have a strong preference for Leucine at P9. The mutation may be a substitution to an amino acid that is able to form a disulphide bond, such as the natural amino acid cysteine or alternatively the substitution may be to a non-natural amino acid that is able to form a disulphide bond.

At least one of the amino acids substituted for an amino acid residue in the F pocket of the MHC binding groove and/or the C terminal anchor residue of the peptide may be a non-natural amino acid, which may be a non-natural amino acid that is able to form a disulphide bond. It is preferred if the C terminal anchor residue of the peptide is so substituted.

Examples of preferred non-natural amino acids that are able to form a disulphide bond include homocysteine and analogues of homocysteine that have an extended carbon side chain, incorporating additional (e.g. one or two) methyl groups. Preferred examples include 2-amino-5-sulfanyl-pentanoic acid, referred to as “h3C” herein (such as provided by Chem-Impex International Inc. Cat No 29777 or Iris Biotech GmbH Cat. No. #917883-62-6), and 2-amino-6-sulfanylhexanoic acid, referred to as “h4C” herein (which can be obtained as a custom synthesis from Creative Chemistry Solutions for example). Non-natural amino acids, including h3C and h4C, may be in D or L isomer configuration.

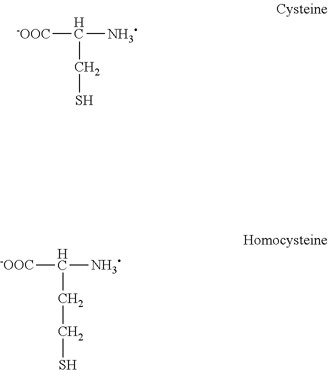

The following show the respective chemical structures of homocysteine, h3C and h4C:

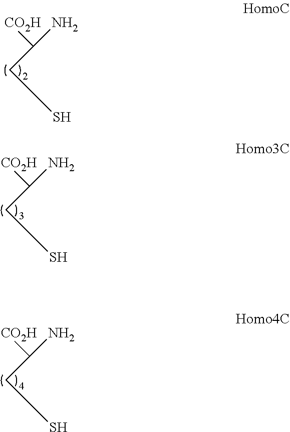

The extended carbon side chains of h3C and h4c mean that the disulphide bridge formed between it and a cysteine residue is increased in length. The following is a schematic representation showing the increased length of the disulphide bridge formed between H3C or H4C and cys, relative to cys-cys.

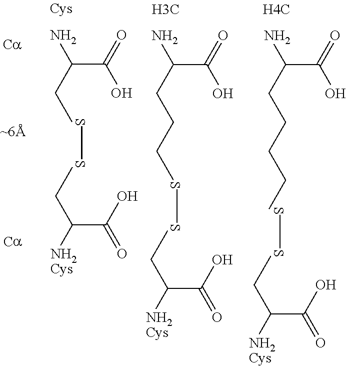

It is preferred if the C terminal anchor residue of the peptide is substituted to one of these non-natural amino acids that are able to form a disulphide bond (preferably h3C or h4C) and the amino acid in the F pocket of the MHC binding groove (preferably residue 116 or 147) is substituted to cysteine.

Given this information, a person skilled in the art can determine which non-natural amino acid (h3C or h4C) is to be substituted into the C terminal anchor residue of the peptide and which residue in the F pocket of the MKC binding groove (116 or 147) is to be substituted to cysteine. It is preferred if the disulphide bond is formed between h3C at the C terminal anchor position of the peptide and a cysteine at position 147 of HLA-E, or between h4C at the C terminal anchor position of the peptide and a cysteine at position 116 of HLA-E. Alternatively, the disulphide bond is formed between h3C at the C terminal anchor position of the peptide and a cysteine at position 116 of HLA-E, or between h4C at the C terminal anchor position of the peptide and a cysteine at position 147 of HLA-E.

Methods for the production of pMHC complexes are known in the art. Typically, MHC complexes are produced recombinantly in a bacterial expression system and refolded in vitro with synthetic peptide. A suitable method is provided in Garboczi et al., (Proc Natl Acad Sci USA. 1992 Apr. 15; 89(8):3429-33) and further described in Example 1 herein. The MHC binding peptide may be produced synthetically, meaning that it is synthesised chemically. Methods for the production of synthetic peptides are known in the art in particular solid phase peptide synthesis (SPPS) also known as Merrifield synthesis.

The complex of the invention may be isolated and/or in a substantially pure form. For example, the complex may be provided in a form which is substantially free of other polypeptides or proteins. The pMHC complexes may be in soluble form, meaning that the MHC complex may be truncated to remove the transmembrane and cytoplasmic regions thereof.

pMHC complexes of the present invention may be further modified. For example, they may be fused to a therapeutic moiety, and/or attached to a solid support, and/or fused to tag, such as a biotin tag, and/or in multimeric form. The tag may be C terminal.

pMHC complexes of the present invention may be associated (covalently or otherwise) with a moiety capable of eliciting a therapeutic effect. Such a moiety may be a carrier protein which is known to be immunogenic. KLH (keyhole limpet hemocyanin) and bovine serum albumin are examples of suitable carrier proteins used in vaccine compositions. Alternatively, the polypeptides and/or polypeptide-MHC complexes of the invention may be associated with a fusion partner. Fusion partners may be used for detection purposes, or for attaching said complex to a solid support, or for pMHC oligomerisation. The pMHC complexes may incorporate a biotinylation site to which biotin can be added, for example, using the BirA enzyme (O'Callaghan et al., 1999 Jan. 1; 266(1):9-15). Other suitable fusion partners include, but are not limited to, fluorescent, or luminescent labels, radiolabels, nucleic acid probes and contrast reagents, antibodies, or enzymes that produce a detectable product.

Detection methods may include flow cytometry, microscopy, electrophoresis or scintillation counting. Fusion partners may include cytokines, such as interleukin 2, interferon alpha, and granulocyte-macrophage colony-stimulating factor.

Isolated peptide MHC complexes may be immobilised by attachment to a suitable solid support. Examples of solid supports include, but are not limited to, a bead, a membrane, sepharose, a magnetic bead, a plate, a tube, a column. Peptide-MHC complexes may be attached to an ELISA plate, a magnetic bead, or a surface plasmon resonance biosensor chip. Methods of attaching peptide-MHC complexes to a solid support are known to the skilled person, and include, for example, using an affinity binding pair, e.g. biotin and streptavidin, or antibodies and antigens. In a preferred embodiment peptide-MHC complexes are labelled with biotin and attached to streptavidin-coated surfaces.

In a second aspect, the present invention provides a multimer of a complex of the first aspect.

pMHC complexes of the invention may be in multimeric form, for example, dimeric, or tetrameric, or pentameric, or octomeric, or greater. Examples of suitable methods for the production of multimeric peptide MHC complexes are described in Greten et al., Clin. Diagn. Lab. Immunol. 2002 March; 9(2):216-20 and references therein and in Wooldridge et al., Immunology (2009) 126(2):147-64). In general, peptide-MHC multimers may be produced using peptide-MHC tagged with a biotin residue and complexed through fluorescent labelled streptavidin. Alternatively, multimeric polypeptide-MHC complexes may be formed by using immunoglobulin as a molecular scaffold. In this system, the extracellular domains of MHC molecules are fused with the constant region of an immunoglobulin heavy chain separated by a short amino acid linker. Polypeptide-MHC multimers have also been produced using carrier molecules such as dextran (WO02072631). Multimeric peptide MHC complexes can be useful for improving the detection of binding moieties, such as T cell receptors, which bind said complex, because of avidity effects.

In a third aspect, the present invention provides a method of making a stabilised pMHC complex of the first aspect, comprising forming a non-native linkage between the C terminal amino acid anchor residue of the peptide, and an amino acid residue in the F pocket of the MHC binding groove.

A convenient way of producing the MHCs useful in the present invention is to express nucleic acid encoding them, by use of nucleic acid in an expression system. The present invention further provides an isolated nucleic acid encoding a stabilised MHC useful in the present invention. Nucleic acid includes DNA and RNA. The skilled person will be able to determine substitutions, deletions and/or additions to such nucleic acids which will still provide the stabilised MHCs useful in the present invention.

The present invention also provides constructs in the form of plasmids, vectors, transcription or expression cassettes which comprise at least one nucleic acid as described above. The present invention also provides a recombinant host cell which comprises one or more constructs as above. As mentioned, a nucleic acid encoding a stabilised MHC useful in the invention forms an aspect of the present invention, as does a method of production of the stabilised MHC which method comprises expression from encoding nucleic acid therefor. Expression may conveniently be achieved by culturing under appropriate conditions recombinant host cells containing the nucleic acid. Following production by expression the stabilised MHC may be isolated and/or purified using any suitable technique, then used as appropriate. Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coll. Expression in prokaryotic cells such as E. coli is well established in the art.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g., ‘phage, or phagemid, as appropriate. For further details see, for example, Sambrook, et al, 1989. Molecular Cloning: a laboratory manual (Cold Spring Harbor, N.Y.: Cold Spring Harbor Laboratory, 1989). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al., Short protocols in molecular biology: a compendium of methods from Current protocols in molecular biology (Brooklyn, N.Y.: Green Pub. Associates: New York, N.Y.: Wiley).

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene. The nucleic acid of the invention may be integrated into the genome (e.g., chromosome) of the host cell. Integration may be promoted by inclusion of sequences which promote recombination with the genome, in accordance with standard techniques. The present invention also provides a method which comprises using a construct as stated above in an expression system in order to express the stabilised MHCs above.

The invention also provides a method of screening, comprising
combining a complex of the first aspect with a population of T cell receptors (TCRs), TCR mimic antibodies or T cells; and
identifying TCRs, TCR mimic antibodies or T cells that bind to the complex.

Preferred features of each aspect of the invention are as for each of the other aspects mutatis mutandis. The prior art documents mentioned herein are incorporated to the fullest extent permitted by law.

### EXAMPLES

The invention will now be described with reference to the following non-limiting examples and figures in which:

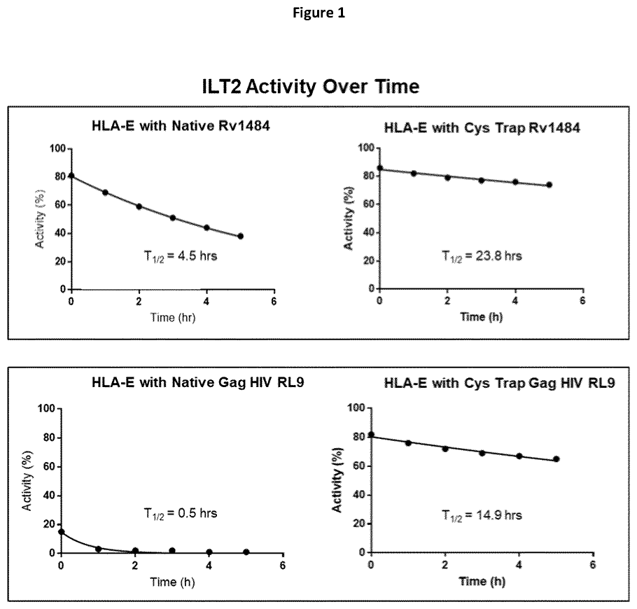

FIG. 1 shows the stability of the indicated pMHC complexes as determined by loss of ILT2 binding over time. Native pHLA-E complexes show limited stability.

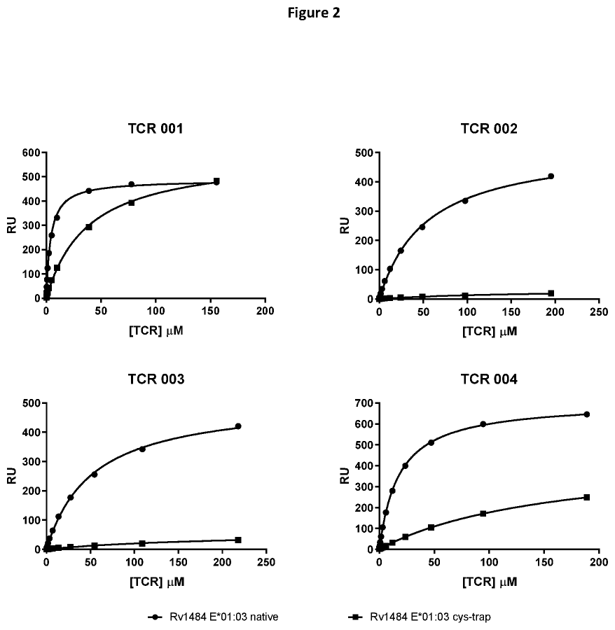

FIG. 2 shows the difference in TCR recognition between a native pMHC complex and the equivalent pMHC complex stabilised using existing methodology.

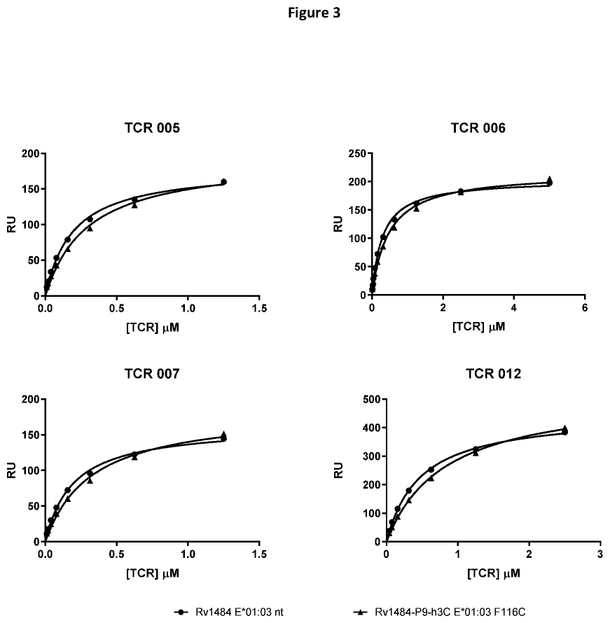

FIG. 3 shows the difference in TCR recognition between a native pMHC complex and the equivalent stabilised pMHC complex of the invention.

### EXAMPLE 1—ISOLATED PEPTIDE HLA-E COMPLEXES HAVE LIMITED STABILITY

This example demonstrates that isolated peptide HLA-E complexes have a short half-life, which means that they are not sufficiently stable to be used for the identification and characterisation of binding agents, such as TCRs and antibodies. A half-life of at least 4 h is typically preferred for such purposes and a half-life substantially in excess of this is desirable.

Stability was assessed using a number of peptides that are known to be presented by HLA-E, including MTB and HIV peptides described in Joosten et al., (PLoS Pathog. 2010 Feb. 26; 6(2):e1000782) and Hansen et al., (Science. 2016 Feb. 12; 351(6274):714-20), respectively, as well as two self peptides corresponding to leader peptides from HLA-A*02 and HLA-Cw3.

### Methods

Peptides

Peptides were obtained by chemical synthesis from Peptide Protein Research Ltd and solubilised in DMSO to a concentration of 4 mg/ml prior to use.

Production of HLA-E*01:01 and HLA-E*01:03 Peptide Complex

HLA-E heavy chain and beta 2-microglobulin (β2m) were expressed separately in E. coli as inclusion bodies and subsequently refolded and purified using previously described methods (Garboczi et al., Proc Natl Acad Sci USA. 1992 Apr. 15; 89(8):3429-33). The HLA-E heavy chain contained a C-terminal biotinylation tag (AviTag™ GLNDIFEAQKIEWHE) and excluded the transmembrane and cytoplasmic domains. Briefly, HLA-E heavy chain and β2m were mixed and refolded together with the peptide of interest, at a ratio of 30:5:2. The soluble refolded pHLAs were then purified using a two-step protocol incorporating anion exchange, followed by size exclusion chromatography (SEC)). To produce biotinylated complexes, a biotinylation step was included following anion exchange and prior to SEC using Biotin-protein ligase (BirA) as described in O'Callaghan et al., Anal Biochem. 1999 Jan. 1; 266(1):9-15.

Sequence of HLA-E*01:03 Heavy chain native +
AviTag™ and F116 highlighted
MGSHSLKYFHTSVSRPGRGEPRFISVGYVDDTQFVRFDNDAASPRMVP
RAPWMEQEGSEYWDRETRSARDTAQIFRVNLRTLRGYYNQSEAGSHTL
QWMHGCELGPDGRFLRGYEQFAYDGKDYLTLNEDLRSWTAVDTAAQIS
EQKSNDASEAEHQRAYLEDTCVEWLHKYLEKGKETLLHLEPPKTHVTH
HPISDHEATLRCWALGFYPAEITLTWQQDGEGHTQDTELVETRPAGDG
TFQKWAAVVVPSGEEQRYTCHVQHEGLPEPVTLRWKPGSGGGLNDIFE
AQKIEWHE
Sequence of HLA-E*01:03 Heavy chain native +
AviTag™ and S147 highlighted
MGSHSLKYFHTSVSRPGRGEPRFISVGYVDDTQFVRFDNDAASPRMVP
RAPWMEQEGSEYWDRETRSARDTAQIFRVNLRTLRGYYNQSEAGSHTL
QWMHGCELGPDGRFLRGYEQFAYDGKDYLTLNEDLRSWTAVDTAAQIS
EQKSNDASEAEHQRAYLEDTCVEWLHKYLEKGKETLLHLEPPKTHVTH
HPISDHEATLRCWALGFYPAEITLTWQQDGEGHTQDTELVETRPAGDG
TFQKWAAVVVPSGEEQRYTCHVQHEGLPEPVTLRWKPGSGGGLNDIFE
AQKIEWHE

The sequence of the AviTag™ and its GSGG linker is underlined and F116 and 5147 are shown in bold and underlined.

Sequence of Human beta-2 microglobulin
MIQRTPKIQVYSRHPAENGKSNFLNCYVSGFHPSDIEVDLLKNGERIEKV
EHSDLSFSKDWSFYLLYYTEFTPTEKDEYACRVNHVTLSQPKIVKWDRDM

Assessment of Peptide-HLA Complex Stability

The stability of peptide-HLA-E complexes was assessed by Surface Plasmon Resonance (SPR) using a BIAcore T200 instrument. Approximately 500-1000 response units (RUs) of purified biotinylated peptide-HLA-E monomers were captured onto streptavidin-coupled CM-5 Series S sensor chips. A soluble, affinity enhanced, form of Ig-like transcript 2 receptor (ILT2) at a concentration of 1 μM was flowed over the surface of the chip at a flow rate of 10 μl min⁻¹ for 60 seconds. ILT2 binds to class I HLA molecules in a conformationally dependent manner and is therefore used as an indicator of complex stability. ILT2 binding to pHLA-E complexes was measured at regular intervals over a time course of 5 hours and responses were then normalised for ILT2 binding by subtracting the bulk buffer response on a control flow cell containing no peptide-HLA. Binding half-life (T_{1/2}) was calculated by plotting % activity against time, using the BIA T200 evaluation software and GraphPad Prism 8.

### Results

Table 1 below provides the half-life (T_{1/2}) for each of the indicated peptides in complex with HLA-E*01:03, as determined by ILT2 binding. All of the complexes have a half-life of under 5 h and several complexes have a half-life of less than 1 h. Representative examples of binding data are provided in FIG. 1 (left-hand panels).

TABLE l

| Peptide origin | Peptide sequence | T _{1/2} (h) (HLA-E*01:03) |
|---|---|---|
| MTB (Rv1484) | RLPAKAPLL | 4.50 |
| MTB (Rv3823c) | ILPSDAPVL | 0.70 |
| MTB (Rv 1518) | VMATRRNVL | 0.57 |
| HIV (Gag 275) | RMYSPTSIL | 0.50 |
| HIV (Gag 275) | RMYSPVSIL | 0.27 |
| Self (HLA-Cw3) | VMAPRTLIL | 3.20 |
| Self (HLA-A*02) | VMAPRTLVL | 3.00 |

These data indicate that native peptide-HLA-E complexes have limited stability, which is unsuitable for identification and characterisation of binding agents.

### EXAMPLE 2—PEPTIDE HLA-E COMPLEXES CAN BE STABILISED VIA CYS TRAP METHODOLOGY BUT DEMONSTRATE PERTURBED TCR BINDING

### A)

In this example, the HLA-E heavy chain was modified to incorporate a cysteine mutation at position Y84; and the peptide was modified to include three additional amino acids (Gly-Cys-Gly) at the C terminus. This approach is commonly referred to as ‘Cys trap’ and has been used successfully to improve the stability of various HLA complexes by ‘trapping’ the peptide in the binding groove (as described in Truscott J Immunol. 2007 May 15; 178(10):6280-9; Mitaksov et al., Chem Biol. 2007 August; 14(8):909-22).

### Methods

The same experimental methods as described in Example 1 were used.

### Results

Table 2 below provides the half-life for each of the indicated cys trapped peptide HLA-E complexes, as determined by ILT2 binding. All of the complexes have a substantially extended half-life compared to the unmodified complexes shown in Example 1, with the majority in excess of 20 h. Representative examples of binding data are provided in FIG. 1 (right-hand panels).

TABLE 2

| Peptide origin | Peptide sequence | T _{1/2} (h) (HLA-E*01:03) |
|---|---|---|
| MTB (Rv 1484) | RLPAKAPLL | 23.8 |
| MTB (Rv 3823c) | ILPSDAPVL | 55.3 |
| MTB (Rv 1518) | VMATRRNVL | 34.0 |
| HIV (Gag 275) | RMYSPTSIL | 14.9 |
| HIV (Gag 275) | RMYSPVSIL | 16.6 |
| Self (HLA-Cw3) | VMAPRTLIL | 27.2 |
| Self (HLA-A*02) | VMAPRTLVL | 48.3 |

These data indicate that modified peptide-HLA-E complexes incorporating a cys trap have improved stability.

### B)

Cys-trap stabilised peptide-HLA-E complex comprising the MTB peptide RLPAKAPLL+GCG was subsequently tested for recognition by antigen specific TCRs and compared to the unmodified complex. This peptide was chosen since the unmodified native peptide HLA-E complex has a relatively long half-life and is therefore amenable to assessment of TCR binding.

### Methods

Assessment of TCR Binding to Peptide-HLA-E Complex

Four TCRs that recognise MTB peptide RLPAKAPLL HLA-E complex were isolated from naïve phage libraries and prepared as soluble alpha beta heterodimers as previously described (Boulter et al., Protein Eng. 2003 September; 16(9):707-11).

Binding Characterisation

Binding analysis of purified soluble TCRs to peptide-HLA complexes was carried out by surface plasmon resonance (SPR), using a BIAcore T200 instrument. Biotinylated pHLA-E molecules were refolded with the peptide of interest as described in Example 1 above. All measurements were performed at 25° C. in Dulbecco's PBS buffer, supplemented with 0.005% surfactant P20.

Biotinylated peptide-HLA-E monomers were immobilized onto streptavidin-coupled CM-5 Series S sensor chips. Equilibrium binding constants were determined using serial dilutions of soluble TCR injected at a constant flow rate of 10-30 μl min⁻¹ over a flow cell coated with ˜1000 response units (RU) of peptide-HLA-E*01:03 complex. Equilibrium responses were normalised for each TCR concentration by subtracting the bulk buffer response on a control flow cell containing no peptide-HLA. The K_{D} value was obtained by non-linear curve fitting using GraphPad Prism 8 software and the Langmuir binding isotherm, bound=C*Max/(C+KD), where “bound” is the equilibrium binding in RU at injected TCR concentration C and Max is the maximum binding.

For high affinity interactions, binding parameters were determined by single cycle kinetics analysis. Five different concentrations of soluble TCR were injected over a flow cell coated with ˜50-200 RU of peptide-HLA complex using a flow rate of 50-60 μl min⁻¹. Typically, 60-200 μl of soluble TCR or was injected at a top concentration of between 100-1000 nM with successive 2 fold dilutions used for the other four injections. The lowest concentration was injected first. To measure the dissociation phase, buffer was injected until 10% dissociation occurred, typically after 1-3 hours. Kinetic parameters were calculated using the BIAevaluation® or BIAcore T200 Evaluation software. The dissociation phase was fitted to a single exponential decay equation enabling calculation of half-life. The equilibrium constant K_{D} was calculated from k_{off}/kₒₙ.

### Results

The binding affinities given in Table 3 below, along with the binding curves shown in FIG. 2, show that while the antigen specific TCRs are able to recognise the native, non-stabilised peptide HLA-E complex, recognition of the cys trap stabilised complex is substantially reduced and in some cases below the level of detection.

TABLE 3

| | K_{D} (μM) | | | |
|---|---|---|---|---|
| | TCR001 | TCR002 | TCR003 | TCROO4 |
| Native (non-stabilised) | 3.97 | 53.69 | 52.07 | 17.78 |
| Cys trap stabilised | 38.02 | ND | ND | 159.9 |

ND - low levels of binding, kinetic parameters could not be determined.

These data indicate that, although the cys trap approach produces stabilised complex, it also perturbs TCR recognition. This may be due to structural alterations of the peptide or MHC resulting from incorporation of the additional residues and formation of the disulphide bond. This approach is therefore not suitable for the production of stabilised peptide HLA-E complexes for the identification and characterisation of binding agents such as TCRs.

### EXAMPLE 3—PRODUCTION OF STABLE PEPTIDE HLA-E COMPLEXES WITH MINIMAL ALTERNATION OF TCR RECOGNITION

### A)

In this example, the peptide HLA-E complex was modified to incorporate a novel engineered disulphide bond between the peptide binding groove of the HLA-E heavy chain and the C terminal anchor residue of the peptide.

To create the novel disulphide, the P9 anchor residue of the MTB peptide RLPAKAPLL peptide was modified to the non-natural amino acid L-3-C homocysteine (2-amino-5-sulfanyl-pentanoic acid) (RLPAKAPL-h3C), and the HLA-E heavy chain was mutated to cysteine at either position F116 or position S147.

### Methods

Peptide HLA-E complexes were prepared and assessed for stability as described in Example 1. TCR binding was assessed as described in Example 2.

### Results

Table 4 below demonstrates that the novel disulphide resulted in a substantial improvement in stability as indicated by the longer half-life of the complex relative to the native complex

TABLE 4

| | T_{1/2} (h) peptide |
|---|---|
| Stabilisation method | HLA-E complex |
| Native (non-stabilised) | 4.5 |
| Non-native disulphide (F116) | 12.3 |
| Non-native disulphide (S147) | 16.6 |

To demonstrate that novel stabilised peptide-HLA-E complexes retain native-like TCR recognition, binding was assessed for 9 different TCRs that had been isolated from a phage library for improved recognition of peptide (RLPAKAPLL)-HLA-E complex. In each case, the kinetics of TCR binding to the stabilised complex was compared to the native complex.

Table 5 and 6 show that TCR binding to the stabilised complex (with a cysteine mutation at F116 or S147 respectively) is preserved in all cases. For each TCR only a small difference in binding is observed between the stabilised and native complex, indicating that the peptide is adopting a near native-like conformation.

FIG. 3 shows the binding curves for four of the TCRs in Table 5.

TABLE 5

| | Native | Non-native | |
|---|---|---|---|
| | complex | disulphide (F116) | Fold |
| TCR | K_{D} (nM) | K_{D} (nM) | difference |
| 005 | 198 | 299 | 1.51 |
| 006 | 294 | 466 | 1.58 |
| 007 | 195 | 325 | 1.66 |
| 009 | 31.1 | 69.7 | 2.24 |
| 010 | 0.822 | 1.6 | 1.95 |
| 011 | 1877 | 2537 | 1.35 |
| 012 | 472 | 820 | 1.73 |
| 013 | 1372 | 1887 | 1.37 |
| 014 | 163 | 258 | 1.58 |

TABLE 6

| | Native | Non-native | |
|---|---|---|---|
| | complex | disulphide (S147) | Fold |
| TCR | K_{D} (nM) | K_{D} (nM) | difference |
| S2a23bwt | 1753 | 1346 | 1.30 |
| S2a15bwt | 462 | 343 | 1.35 |
| S2a25bwt | 97.6 | 81.2 | 1.20 |
| S2a24b07 | 6280 | 5360 | 1.17 |
| S2a22b07 | 765 | 681 | 1.12 |

### B)

In a further example, the P9 anchor residue of the MTB peptide RLPAKAPLL peptide was modified to the non-natural amino acid L-4-C homocysteine (2-amino-6-sulfanylhexanoic acid) (RLPAKAPL-h4C), and the HLA-E heavy chain was mutated to cysteine at position F116 or S147. Complex stability and TCR binding were assessed as described in part A.

### Results

The binding half life of the resulting complex was 24.47h, demonstrating that the novel disulphide resulted in a substantial improvement in stability relative to the native complex (as shown in Table 4). TCR binding was assessed for 6 TCRs. In all 6 cases, TCR binding to the stabilised complex was preserved. The difference in binding relative to the native complex ranged from 1.53 to 3.24 fold for the disulphide with F116 and from 1.11 to 2.83 fold for the disulphide with S147.

TABLE 7

| | Native | Non-native | Non-native | | |
|---|---|---|---|---|---|
| | complex | disulphide | disulphide | Fold | Fold |
| | K_{D} | (F116) | (S147) | difference | difference |
| TCR | (nM) | K_{D} (nM) | K_{D} (nM) | (F116) | (S147) |
| 1 | 3.88 | 12.57 | 10.97 | 3.24 | 2.83 |
| 2 | 53.69 | 124.00 | 95.86 | 2.31 | 1.79 |
| 3 | 3.01 | 8.13 | 7.38 | 2.70 | 2.45 |
| 4 | 0.48 | 1.34 | 0.99 | 2.80 | 2.08 |
| 5 | 1.36 | 2.42 | 1.57 | 1.78 | 1.15 |
| 6 | 0.67 | 1.03 | 0.75 | 1.53 | 1.11 |

1. A stabilized peptide-MHC (pMHC) complex, comprising a non-native linkage between the C terminal anchor residue of the peptide, and an amino acid residue in the F pocket of the MHC binding groove.

2. The complex of claim 1, wherein the non-native linkage is a covalent bond.

3. The complex of claim 2, wherein the covalent bond is formed between amino acids substituted for amino acid residues in the F pocket of the MHC binding groove and/or the C terminal anchor residue of the native peptide, preferably in both the F pocket of the MHC binding groove and the C terminal anchor residue of the native peptide.

4. The complex of claim 3, wherein the substituted amino acid residue in the F pocket of the MHC binding groove is at position 116 or 147.

5. The complex of claim 1, wherein the non-native linkage is a disulfide bond.

6. The complex of claim 5, wherein the amino acid residue at position 116 or 147 of the MHC heavy chain is substituted to cysteine.

7. The complex of claim 3, wherein the amino acid substituted for the C-terminal anchor residue of the peptide is a non-natural amino acid.

8. The complex of claim 7, wherein the C terminal amino acid anchor residue of the peptide is substituted to an analogue of homocysteine that has an extended carbon side chain.

9. The complex of claim 8, wherein the analogue of homocysteine is 2-amino-5-sulfanyl-pentanoic acid or 2-amino-6-sulfanylhexanoic acid.

10. The complex of claim 1, wherein the complex is soluble.

11. The complex of claim 1, wherein the MHC includes a biotin tag, optionally wherein the tag is C terminal.

12. The complex of claim 1, wherein the MHC is HLA-E.

13. A multimer of the complex of claim 1.

14. A method of making the peptide-MHC complex of claim 1, comprising forming a covalent bond between the MHC heavy chain and the C terminal amino acid anchor residue of the peptide.

15. A method of screening, comprising
combining the complex of claim 1 with a population of T cell receptors (TCRs), TCR mimic antibodies or T cells; and
identifying TCRs, TCR mimic antibodies or T cells that bind to the complex.
